(19) United States
(12) Patent Application Publication
(10) Pub. No.: US 2024/0189620 A1
(43) Pub. Date: Jun. 13, 2024

(54) DEVICE AND ASSOCIATED METHODS FOR PRECISION RADIATION TREATMENT

(71) Applicants: Arizona Board of Regents on Behalf of the University of Arizona, Tucson, AZ (US); Dignity Health, San Francisco, CA (US)

(72) Inventors: Jennifer Kehlet Barton, Tucson, AZ (US); Nitika Thawani, San Francisco, CA (US); Danielle Ann Gelb, Tucson, AZ (US); Audrey Tamra Cohen, Tucson, AZ (US); Hiram Olivas, Tucson, AZ (US); Jacob Emanuel Mapp, Tucson, AZ (US); Ryan Stephen Zenhausern, Tucson, AZ (US); Jesus Daniel Vargas Lopez, Tucson, AZ (US)

(21) Appl. No.: 18/553,713

(22) PCT Filed: Apr. 28, 2022
(86) PCT No.: PCT/US2022/026824

§ 371 Date: Oct. 2, 2023

Related U.S. Application Data
(60) Provisional application No. 63/180,985, filed on Apr. 28, 2021.

(51) Int. Cl. A61N 5/10; A61M 25/09; A61M 25/10

(52) U.S. Cl. CPC A61N 5/1014; A61M 25/09; A61M 25/1011; A61N 2005/1003; A61N 2005/1008; A61N 2005/1018

(57) ABSTRACT

A system includes a device associated with an endoscopy guided procedure including a housing defining a housing channel with at least a portion of the housing configured for deployment within a cavity. The device further includes a plurality of bodies defining respective channels defined within the housing. The housing defines a longitudinal axis, and the plurality of channels extends throughout the housing around the longitudinal axis. Each of the plurality of channels accommodates selective activation of radiation emittance from a predetermined different position along the housing via one or more radiation elements such that the plurality of channels collectively provides a predetermined spatial distribution of a radiation treatment to a target area along the cavity.

### CROSS REFERENCE TO RELATED APPLICATIONS

This is a PCT patent application that claims benefit to U.S. provisional application Ser. No. 63/180,985 filed on 28 Apr. 2021 entitled PRECISION BRACHYTHERAPY DEVICE AND ASSOCIATED METHODS FOR TREATMENT OF ESOPHAGEAL CANCER which is incorporated by reference in its entirety.

### FIELD

The present disclosure generally relates to targeted radiation treatment methods, and in particular, to a system including a device and associated methods for more precise radiation treatment with endoscopy-guided radiation applications.

### BACKGROUND

Esophageal cancer is often difficult to treat due to its position within the body and the fact that most patients present with advanced disease. Radiation treatment, or brachytherapy, is often employed through insertion of an endoscope into the esophagus, and then radiation is applied to destroy cancerous tissue. However, predicate devices also damage healthy tissue and accuracy of radiation application is lacking. Current esophageal brachytherapy devices do not differentiate radiation doses between healthy and cancerous tissue, decreasing treatment efficacy.

It is with these observations in mind, among others, that various aspects of the present disclosure were conceived and developed.

### SUMMARY

The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below. Corresponding apparatus, methods/processes, systems, and other implementations are also within the scope of the disclosure.

In one example, the present inventive concept may take the form of a system for precise radiation treatment including a device. The device, associated with an endoscopy guided procedure, includes a housing defining a housing channel extending along a longitudinal axis of the housing with at least a portion of the housing configured for deployment within a cavity. The device further includes a plurality of bodies defining a plurality of channels. The plurality of channels accommodates selective activation of radiation emittance from a predetermined different position along the housing via one or more radiation elements such that the plurality of channels collectively provides a predetermined spatial distribution of a radiation treatment to a target area along the cavity. The system may further include a stabilizing subsystem including one or more balloons in communication with balloon catheters; the one or more balloons arranged along the distal portion of the housing.

In some examples, the system further includes an endoscope, wherein the endoscope assists with endoscopy-guided placement of the device along a gastrointestinal tract. In some examples, the predetermined spatial distribution of the radiation treatment defines a plurality of shape configurations different from general concentric shapes for customizable dose delivery. In some examples, the plurality of channels comprises a first channel configured to receive a first radiation element and emit radiation along a first position relative to the longitudinal axis of the housing and a second channel of the plurality of channels configured to receive a second radiation element and emit radiation along a second position relative to the longitudinal axis. In some examples, the system includes a fluoroscopy subsystem that verifies a predetermined position of one or more radiation elements along one or more of the plurality of channels.

In some examples, the housing is a portion of a brachytherapy device and the cavity is an esophagus of a gastrointestinal tract. In other examples, the metal coating is silver, wherein a 12 um silver deposition is applied to outer surfaces of the plurality of channel that provides 240 degrees of radiation shielding. In some examples, a length of the device is predetermined and suitable for application to an esophagus defined by the cavity.

In some examples, the radiation element includes at least one of a radiation seed, radiation ribbon, or radiation capsule. In some examples, the radiation elements are fed to the device and positioned along any of one or more predetermined dwell points using a cable that traverses along a channel of the plurality of channels. Radiopaque markers viewable during a fluoroscopy can verify positioning of the radiation elements and/or the device generally.

In some examples, the device is positioned along the target area during an endoscopy and the device traverses where an endoscope is applied.

In some examples, the system includes an adapter subsystem in communication with a console that controls movement of one or more radiation seeds through the plurality of channels to apply the radiation treatment.

In some examples, each of the plurality of channels is positioned radially around a guidewire channel extending along the longitudinal axis of the housing and configured to produce the same radiation plot. The plurality of channels is encapsulated by the housing.

In another example, the present inventive concept can take the form of a method of manufacturing a device comprising the steps of forming a device including a housing defining a housing channel extending along a longitudinal axis of the housing and disposing a plurality of bodies defining a plurality of channels within the housing along the longitudinal axis. The method further includes the step of applying a silver coating along outer surfaces of the plurality of bodies to attenuate radiation. The plurality of channels of the present method accommodates selective activation of radiation emittance from a predetermined different position along the housing via one or more radiation elements such that the plurality of channels collectively provides a predetermined spatial distribution of a radiation treatment to a target area along a cavity.

In another example, the present inventive concept can take the form of a method, comprising the steps of positioning a device along a target area, the device including a housing defining a housing channel and a plurality of bodies extending along the housing channel, the plurality of bodies defining a plurality of channels that accommodate selective activation of radiation emittance from a predetermined different position along the housing using one or more radiation elements. The method further includes the step of verifying and/or guiding a position of the device using an endoscope or other such imaging device. The method further includes the step of selectively emitting radiation by engaging a plurality of radiation elements along one or more of the plurality of channels to predetermined dwell positions to define a first spatial distribution of radiation treatment for a first time period. The method further includes the step of verifying predetermined dwell positions of the radiation elements via fluoroscopy. The method further includes the step of selectively emitting radiation by modifying a position and/or number of the plurality of radiation elements along one or more of the plurality of channels to define a second spatial distribution of radiation treatment different from the first spatial distribution for a second time period. The method further includes the step of verifying treatment progress associated with the first spatial distribution and the second spatial distribution of the radiation treatment from the first time period to the second time period.

These examples and features, along with many others, are discussed in greater detail below.

### BRIEF DESCRIPTION OF THE DRAWINGS

FIG. 1 is a simplified block diagram of an example system architecture including an exemplary component hierarchy associated with a device of the system suitable for precision radiation treatment as described herein.

FIG. 2 is simplified system block diagram illustrating relationships between the components of the system of FIG. 1 and other optional components.

FIG. 3 is side view of one example of the device of FIG. 1 and other exemplary components of the system.

FIG. 4 is an exploded isometric view of the device of FIG. 3.

FIG. 5A is an isometric view of an applicator cap of the device of FIG. 3.

FIG. 5B is a cross-sectional plan view of the housing of the device of FIG. 3 that engages with the applicator cap of FIG. 5A.

FIG. 6 is an isometric cross-sectional view of the distal portion of the device of FIG. 3 illustrating further aspects of the device and an exemplary stabilizing subsystem.

FIG. 7 is an isometric view of an end cap of the device of FIG. 3.

FIG. 8 is an enhanced view of a portion of the housing showing the radiopaque markers for device guidance and/or radiation delivery guidance.

FIG. 9A is an isometric cross-sectional view of a portion of the device of FIG. 3 illustrating a general example configuration of the plurality of channels and positioning relative to the stabilizing subsystem and the guidewire subsystem.

FIG. 9B is an isometric cross-sectional view of a portion of the device of FIG. 3 in an example where the plurality of channels includes a metal coating.

FIG. 9C is an isometric view of a portion of a channel of FIG. 9B with the metal coating illustrating aspects of the shielding subsystem.

FIG. 10 is a graph illustrating aspects associated with testing of the device of FIG. 3 and focusing on radiation does at tissue following only R² as described herein.

FIG. 11 is a graph illustrating aspects associated with testing of the device of FIG. 3 and focusing on radiation does at tissue following only Attenuation laws.

FIG. 12 is a graph illustrating aspects associated with testing of the device of FIG. 3 and focusing on radiation dose at tissue following only R² as described herein.

FIG. 13 is a graph illustrating aspects associated with testing of the device of FIG. 3 and focusing on radiation dose at tissue following only R² as described herein.

FIG. 14 is a graph illustrating aspects associated with testing of the device of FIG. 3 and focusing on radiation does at tissue following only Attenuation laws.

FIG. 15 is a graph illustrating aspects associated with testing of the device of FIG. 3 and focusing on final radiation dose prediction at tissue.

FIG. 16 is an exemplary method associated with examples of the system and the device described herein for endoscopy-guided radiation treatment.

FIG. 17 is a cross-sectional view of the device described herein.

FIGS. 18A-18C are illustrations of exemplary phases of a predetermined application of the system and the device described herein for generating one or more different spatial distributions of radiation treatment to a target area.

Corresponding reference characters indicate corresponding elements among the view of the drawings. The headings used in the figures do not limit the scope of the claims.

### DETAILED DESCRIPTION

Various examples of a system including a device and associated methods for precise radiation treatment are disclosed. In general, a system for precise radiation treatment described herein includes a device. The device, associated with an endoscopy guided procedure, includes a housing defining a housing channel extending along a longitudinal axis of the housing with at least a portion of the housing configured for deployment within a cavity. The device further includes a plurality of bodies defining a plurality of channels. The plurality of channels accommodates selective activation of radiation emittance from a predetermined different position along the housing via one or more radiation elements such that the plurality of channels collectively provides a predetermined spatial distribution of a radiation treatment to a target area along the cavity. The system may further include a stabilizing subsystem including one or more balloons in communication with balloon catheters; the one or more balloons arranged along the distal portion of the housing. In some examples, outer surfaces of the plurality of bodies are at least partially covered with a silver plating for radiation attenuation.

The disclosed device and system provide various improvements over endoscopy guided procedures. For example, current esophageal brachytherapy devices do not differentiate radiation dose between healthy and cancerous tissue, decreasing treatment efficacy. The present system is operable to function within a current clinical environment and deliver twice the effective radiation dose to cancerous tissue than to healthy tissue. The present brachytherapy device can be configured to be used in a patient with esophageal cancer of any presentation. In some examples, the device emits two times higher radiation doses to the cancerous tissue than to the healthy esophageal tissue, functions in 95% of all esophageal cancer patients, interfaces with existing brachytherapy equipment, inserts and retracts from the patient without causing bleeding, is stable in the patient, and is a cost-effective, disposable device.

In some examples, the system components and design elements are mechanical and limited to mechanical components in order to minimize the risk of failure and improve device reliability while also keeping device costs to a minimum. The system further includes materials compatible with gamma radiation used during the procedure, ethylene oxide for sterilization of the device, and diluted acid to simulate the conditions of the esophageal tract. The system is compatible with existing brachytherapy equipment and does not require additional training of radiation oncologists or medical physicists prior to use. These design elements ensure a seamless transition from other brachytherapy devices to the device design. In one example, the system includes a silver coating to a plurality of radiation (seed) channels. In some examples, the silver coating is 12 μm in thickness and coats 240 degrees of the seed channel's outer surface. This plating was added to increase the device's ability to attenuate radiation and protect healthy esophageal tissue.

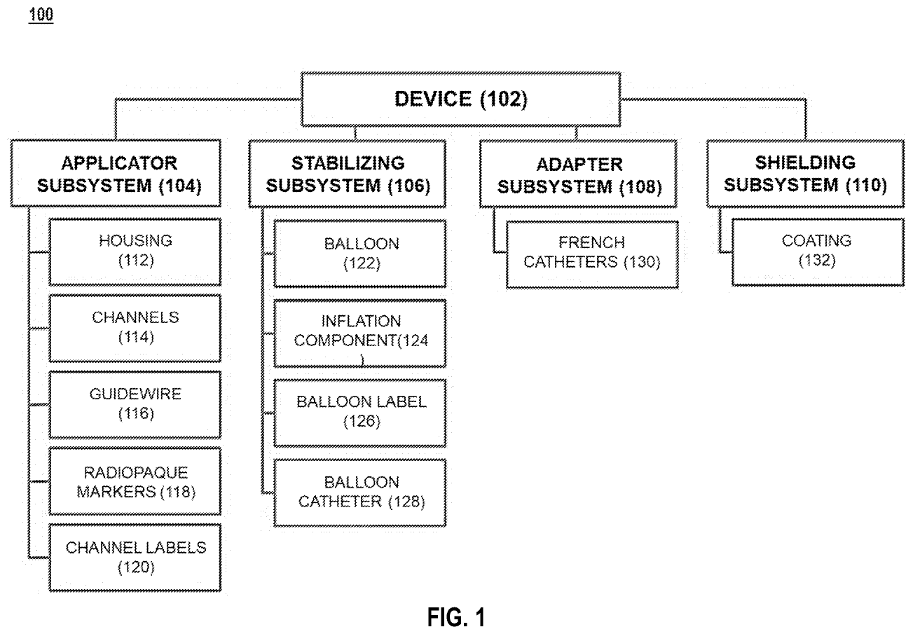

Referring to FIG. 1, a system 100 is shown including a device 102 for precision radiation treatment. In general, the device 102 is suitable for an endoscopy-guided procedure, such as (but not limited to) brachytherapy radiation treatment of esophageal cancer. In addition to the device 102, examples of the system 100 may include an applicator subsystem 104, a stabilizing subsystem 106, an adapter subsystem 108, and a shielding subsystem 110. The applicator subsystem 104 defines exemplary aspects of the device 102, including a housing 112, a plurality of channels 114 defined within the housing 112, and a guidewire 116 (engaged within a guidewire channel 117 and) positioned along the housing 112 as further described herein. In addition, the applicator subsystem 104 may include (one or more) radiopaque markers 118 defined along predetermined positions of the housing 112 to assist with positioning of the device 102 and delivery of treatment (device 102 is viewable under a computed tomography (CT) scan), and channel labels 120 distinguishing each of the plurality of channels 114 and/or providing dwell positions along the housing 112 for radiation treatment as further described herein.

The stabilizing subsystem 106 of FIG. 1 can assist with centralization and stabilization of the device 102 along a target area and in some examples maintains the device 102 in a fixed position relative to a target area. The stabilizing subsystem 106 includes (one or more of) a balloon 122, (one or more of) an inflation component 124 for inflating the balloon 122 optionally including one or more valves and pumps (e.g., Luer-Lock valves), (one or more of) a balloon label 126, and (one or more of) a balloon catheter 128 defined within the housing along the plurality of channels 114. As further shown, the adapter subsystem 108 can include one or more French catheters 130 generally configured to control movement of a radiation element within the housing through the plurality of channels 114 as further described herein. The shielding subsystem 110 includes a coating 132, which may be metal such as silver, that may be applied to one or more of the plurality of channels 114 to reduce radiation application outside of the target area and for radiation attenuation, as further described herein.

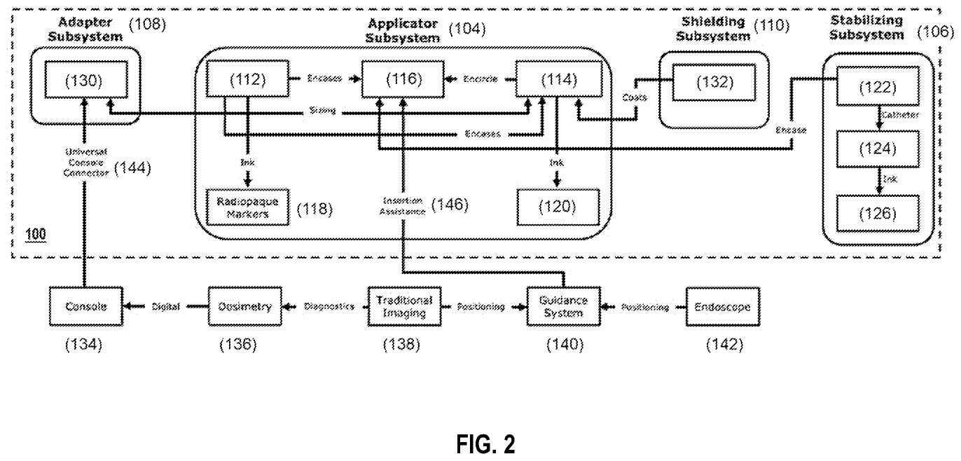

Referring to FIG. 2, the various exemplary subsystems of the system 100 and other optional components can be engaged and otherwise interact as indicated to provide precision radiation treatment as further described herein. As illustrated for example, the coating 132 of the shielding subsystem 110 can be implemented to coat one or more of the plurality of channels 114 of the device 102, and the shielding subsystem 110 as implemented in examples of the device 102 surrounds or encases the guidewire 116 of the applicator subsystem 104. Further, various other components may be implemented with the system 100. For example, a console 134 may be employed in combination with a universal console connector 144 to feed one or more radiation elements to the device 102 (e.g., microSelectron® console including any loading/after-loading device with suitable hardware and software). Dosimetry 136 may be utilized (via one or more dosimeters) to measure, calculate, and assess radiation applied through use of the device 102. Traditional imaging 138 (e.g., computed topography (CT), X-Ray, and the like) may be employed along with one or more of a guidance system 140 to position the device 102 as intended for a given procedure, and an endoscope 142 can be implemented for guidance of the device 102 along a target area for insertion guidance 146 or otherwise. The aforementioned components are merely exemplary, and alternative like components and/or other combinations are contemplated.

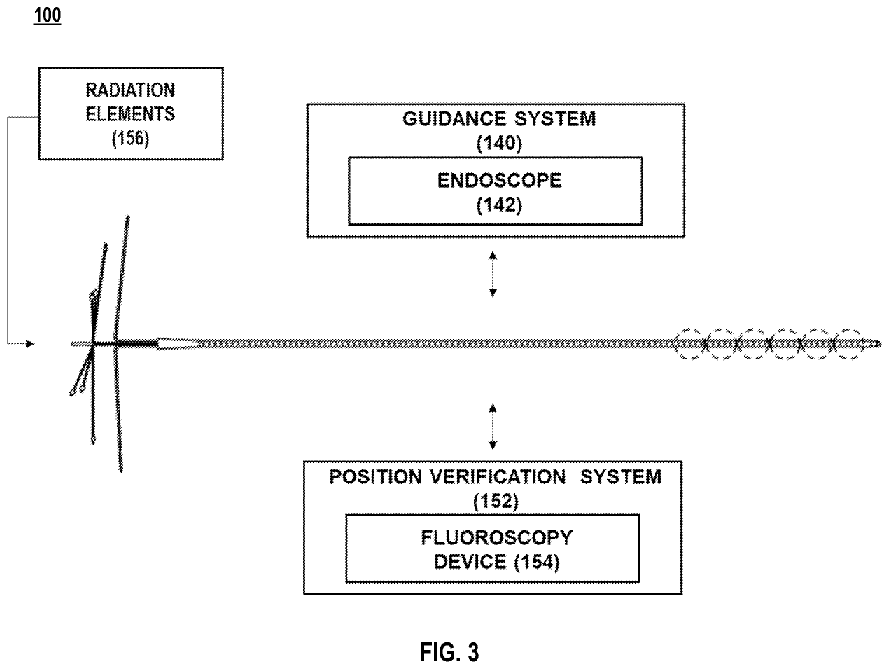

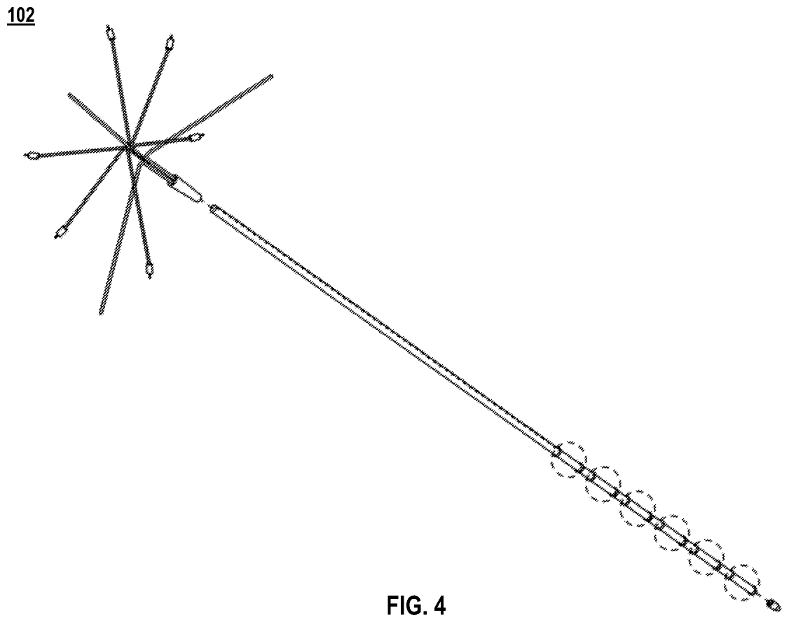

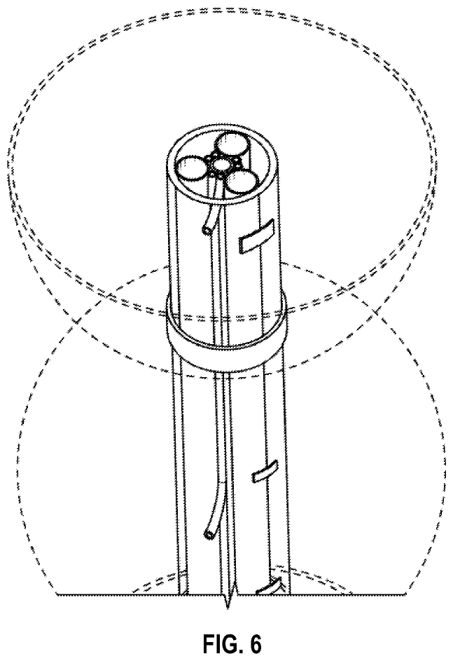

Referring to FIGS. 3-4, additional details associated with the applicator subsystem 104 and the device 102 generally are provided. As indicated, the device 102 includes a body 150 defining a longitudinal axis 151, a proximal portion 153A, and a distal portion 153B. The housing 112 of the body 150 of the device 102 shown may generally be hollow and consequently defines a housing channel 155 (more clearly shown in FIG. 6) from the proximal portion 153A to the distal portion 153B. Encapsulated within the housing 112 is a plurality of bodies 157 defining the plurality of channels 114, and at least one balloon catheter may extend from the proximal portion 153A towards the distal portion 153B within the housing channel 155. The plurality of bodies 157 may include general tubular members, catheters, conduits, or any such structure suitable for receiving and transporting one or more radiation elements (156).

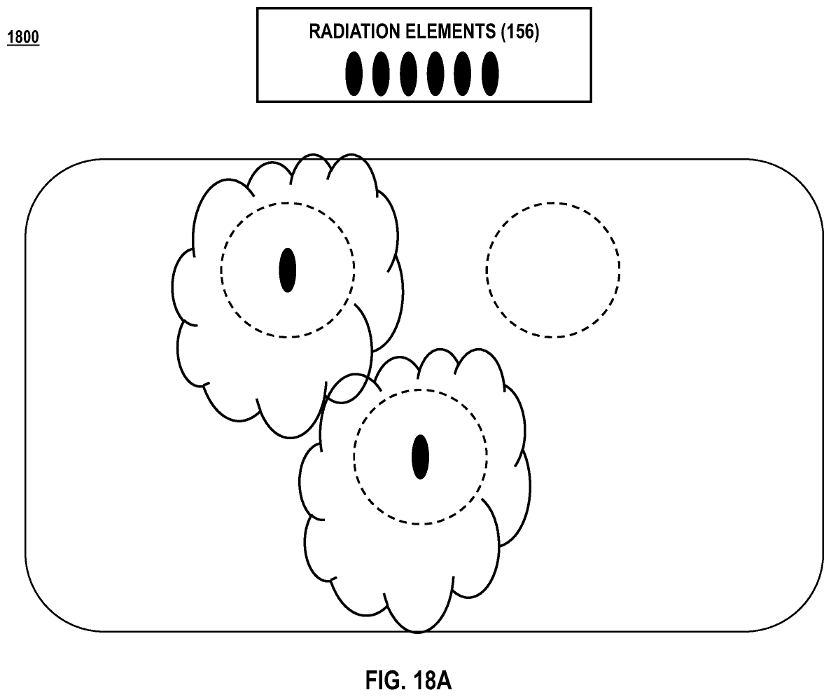

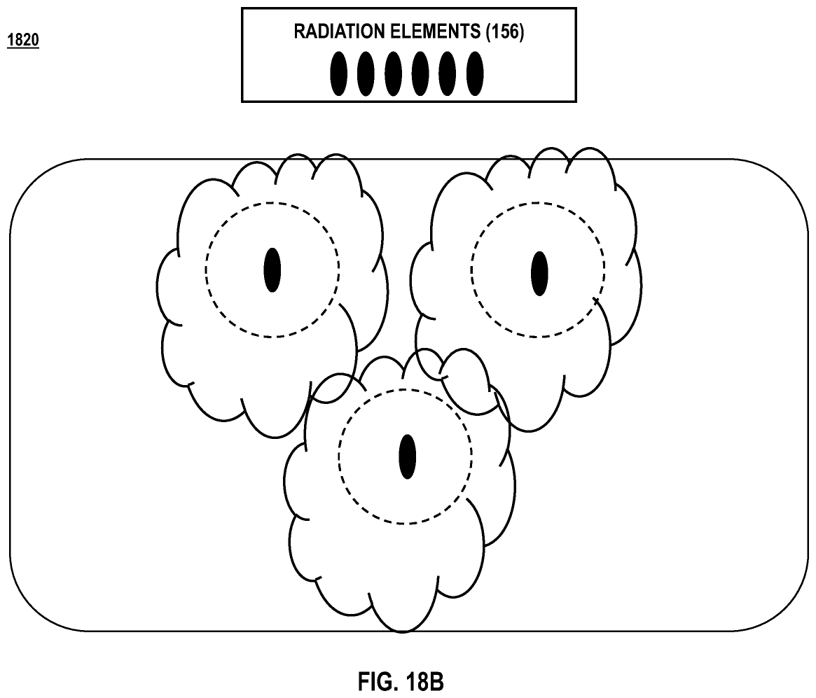

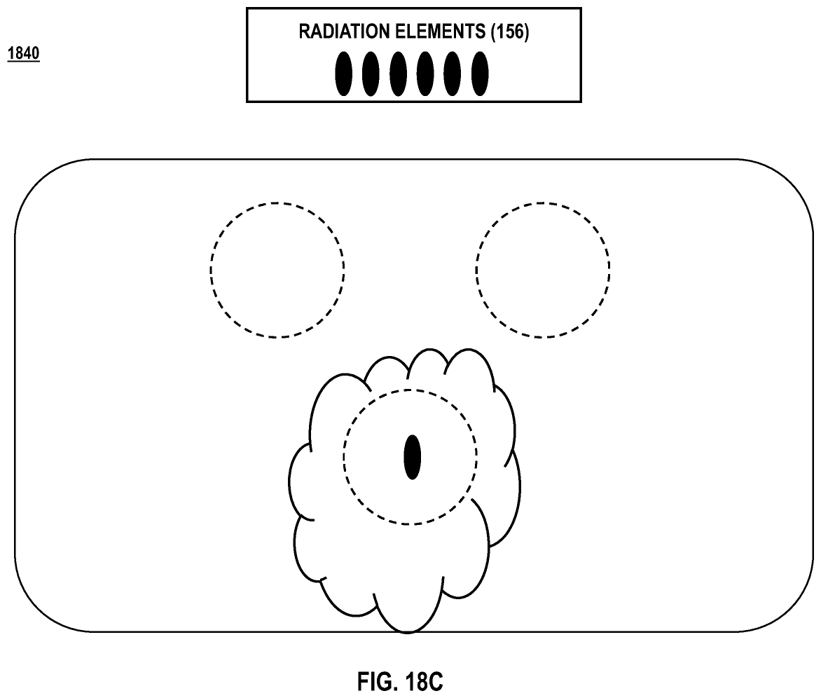

In general, the longitudinal axis 151 is defined longitudinally along a general center portion of the housing 112 (and the housing channel 155) and the plurality of channels 114 extends along the housing channel 155 around the longitudinal axis 151 (shown in FIG. 6). As described herein, each of the plurality of channels 114 defined by the plurality of bodies 157 accommodates selective activation of radiation emittance from a predetermined different position along the housing 112 via one or more radiation elements (FIGS. 18A-18C) such that the plurality of channels 114 collectively provides a predetermined spatial distribution of a radiation treatment to a target area along the cavity (FIGS. 18A-18C). In some examples, the housing 112 comprises a flexible material such that the housing 112 accommodates a predetermined amount of deflection between the proximal portion 153A and the distal portion 153B. In some examples, a length of the device 102 is predetermined to be suitable for application to an esophagus area. The channel labels 120 shown may be positioned anywhere along predetermined locations of the housing 112 to, e.g., distinguish between bodies of the plurality of bodies 157, to identify predetermined dwell positions along respective channels 114 of the bodies 157 for radiation elements 156, and the like.

As further indicated, the device 102 may be deployed in combination with the guidance system 140 of FIG. 2 including an endoscope 142, and/or a position verification system 152 including at least one fluoroscopy device 154. In this manner, the device 102 may be endoscopy-assisted in that the guidance system 140 assists with positioning of the device 102 along a target area (e.g., a gastrointestinal tract). The position verification system 152 can be implemented to verify positioning of the device 102 by way of the radiopaque markers 118 or otherwise and/or may be implemented to verify a final position of one or more radiation elements 156 within one or more of the plurality of channels 114.

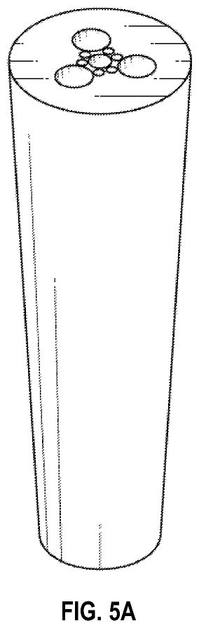

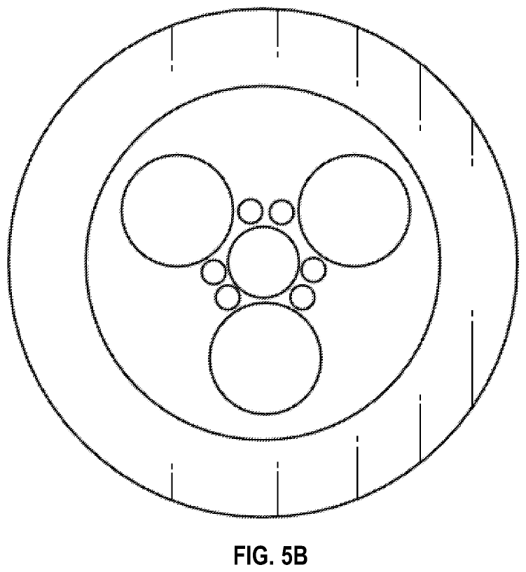

Examples of the device 102 include an applicator cap 158 positioned along the proximal portion 153A of the device 102 and an end cap 160 positioned along the distal portion 153B for engagement with a general terminal end 161 of the device 102. The applicator cap 158 includes a plurality of openings or channels (e.g., channels 162 of FIG. 5B) that align with the plurality of channels 114 and other portions of the housing 112, as further described herein. The end cap 160 encloses the housing 112 along the terminal end 161 and is generally oriented towards a target area during treatment, as further described herein. The end cap 160 may include a beveled shape configuration to facilitate engagement along the target area, such as the esophagus. As further shown, the guidewire 116 is seated within the guidewire channel 117, extends from the proximal portion 153A of the device 102 towards the distal portion 153B, and is generally centered along the longitudinal axis 151 (FIG. 6). The guidewire 116 provides assistance with guiding the housing 112 to a predetermined position relative to a cavity or other target area.

Further, when implemented, the stabilizing subsystem 106 may be generally defined along the distal portion 153B of the device 102 as indicated. In particular, one or more balloons 122 (e.g., medical grade) may be positioned along the distal portion 153B aligned in series or otherwise aligned. In some cases, one or more of the balloons 122 may be threaded over the distal portion 153B of the device 102 as further described herein. In some examples, the stabilizing subsystem 106 includes one or more of the balloon catheters 128 formed within the housing 112 along the longitudinal axis 151 in communication with one or more balloons 122 positioned along the housing 112 as shown. In such examples, each balloon catheter of the one or more balloon catheters 128 is in communication with a respective balloon 122. Further, as previously described, each balloon catheter of the one or more balloon catheters 128 may include an inflation component 124 such as a respective valve for intake and expulsion of air for a respective balloon (122).

Referring to FIGS. 5A, the applicator cap 158 may define a cylindrical or conical shape and may include a plurality of channels 162 configured for alignment with the plurality of channels 114, one or more of the balloon catheters 128, and the guidewire channel 117. More specifically, an example of the applicator cap 158 shown includes a plurality of applicator channels 164 configured for alignment with the plurality of channels 114 of the housing 112, a plurality of channels 165 that align with the balloon catheters 128, and a channel 166 that aligns with the guidewire channel 117 of the housing 112. In some examples, the applicator cap 158 accommodates receipt and initial feeding of the radiation elements 156 into the plurality of channels 114.

As indicated in FIG. 5B, the housing 112 of the body 150 of the device 102 may be defined by an interior surface 171 of the body 150, which is generally cylindrical, defines the housing channel 155, and encapsulates the plurality of bodies 157, the plurality of channels 114, the guidewire 116 and guidewire channel 117, and the one or more balloon catheters 128. As further indicated, the plurality of channels 114 may be spaced equidistantly around the guidewire channel 117 and the longitudinal axis 151. In some examples, as shown, the plurality of bodies 157 defining the plurality of channels 114 includes three separate bodies. When implemented, the balloon catheters 128 may be similarly spaced equidistantly to one another around the longitudinal axis 151 as shown. In some examples, the balloon catheters 128 may be arranged in pairs as shown and deliver air to one or more balloons 122.

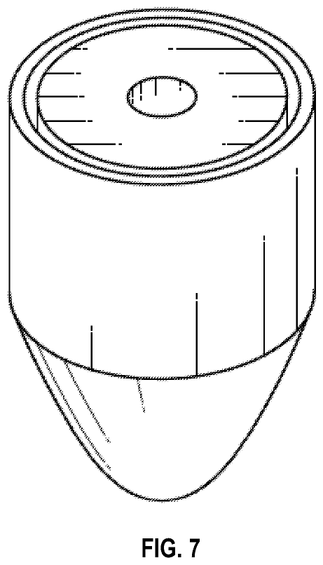

FIG. 6 illustrates the device 102 from the distal portion 153B of the housing 112 and the stabilizing subsystem 106, including the plurality of channels 114, the guidewire channel 117, and the balloon catheters 128 of FIG. 5B shown along the terminal end 161 of the housing 112. As indicated, a balloon catheter 128A is in communication with a respective balloon 122A for inflation and deflation (e.g., by an inflation component 124 shown in FIG. 4 or otherwise). In addition, examples of the radiopaque markers 118 are shown which may be useful for guidance and positioning as described. Turning to FIG. 7, with reference back to FIG. 4, the end cap 160 engages with the distal portion 153B of the housing 112 along the terminal end 161 and defines a central depression 170 that communicates with the guidewire channel 117 to seat a portion of the guidewire 116. In some examples, the end cap 160 includes an engagement portion 172 that mates with the terminal end 161 of the housing (e.g., male and female threads, general snap configuration, etc.). In general, the end cap 160 encloses the housing channel 155 along the distal portion 153B.

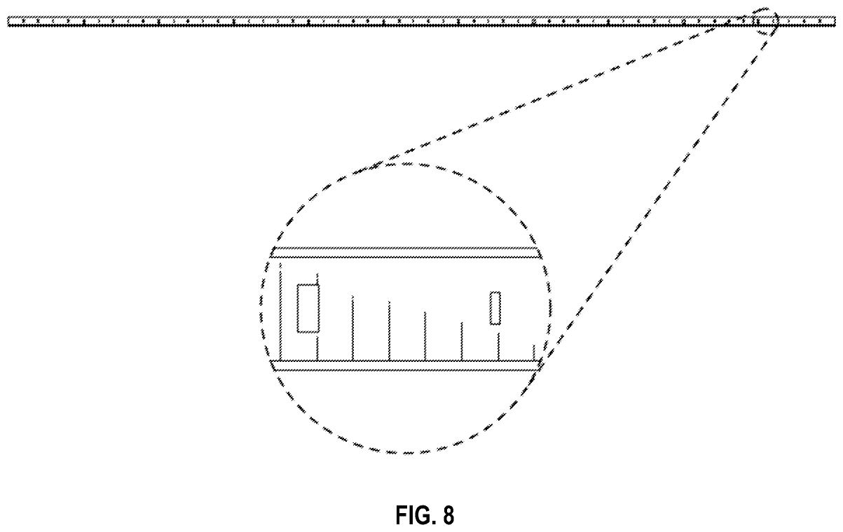

FIG. 8 shows an enhanced region 180 taken along a portion 182 of the housing 112 to illustrate further aspects of the radiopaque markers 118. As indicated, any of the radiopaque markers 118 may be spaced along predetermined positions of the housing 112 to assist with positioning of the device 102 and delivery of treatment, and and/or to identify dwell positions along the housing 112 for radiation treatment. In some examples, the radiopaque markers 118 are placed every 1 cm along the outer surface of the housing 112. In some examples, the outer surface of the housing 112 further includes hash marks 184 to facilitate alignment and positioning of the device 102 along the target area.

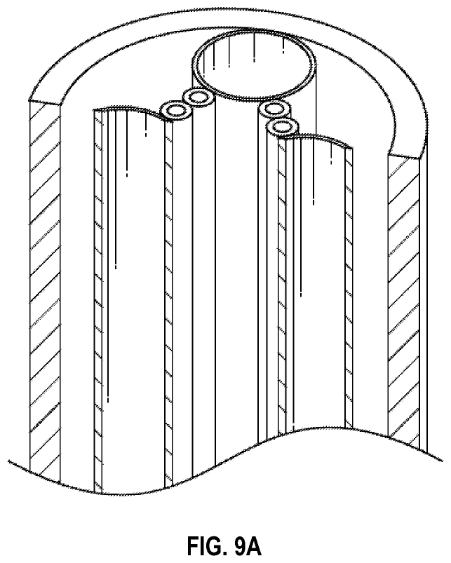

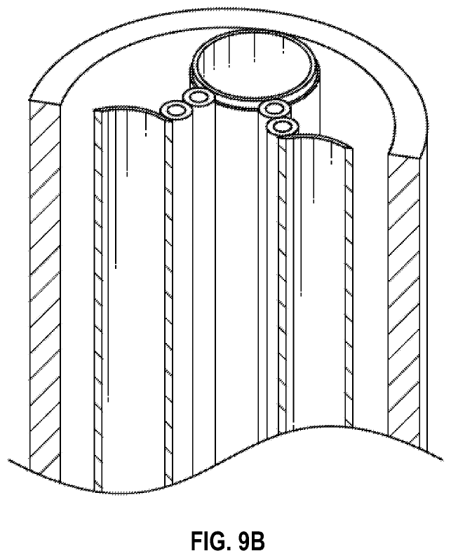

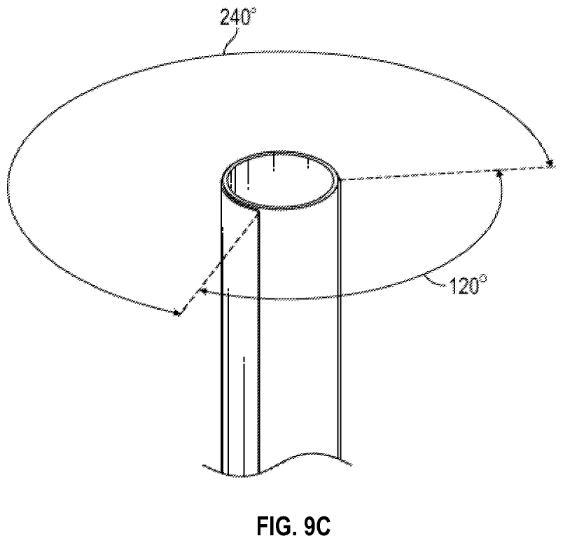

FIGS. 9A-9C illustrate aspects of the shielding subsystem 110 in some examples of the device 102. FIG. 9A in particular shows an example of the device 102 devoid of the shielding subsystem 110. In other words, under this example, the bodies 157 are devoid of the coating 132. In some examples, each of the plurality of channels 114 includes an outer diameter of 2 mm and an inner diameter of 1.85 mm (+/−0.05 mm), each of the balloon catheters 128 includes an outer diameter of 0.52 mm (+/−0.05 mm) and an inner diameter of 0.3 mm (+/−0.1 mm), and the housing 112 includes an outer diameter of 6.5 mm (+/−0.5 mm) and an inner diameter of 5.5 mm (+/−0.5 mm).

By contrast, FIGS. 9B-9C illustrate examples of the device 102 with the coating 132 applied to outer surfaces 187 of the plurality of bodies 157. In these examples where the shielding subsystem 110 and the coating 132 is applied to outer surfaces 187 of the plurality of bodies 157, many variations are contemplated. For example, the coating 132 may be applied to one or more of the plurality of bodies 157, a portion of each of the outer surfaces 187 of the plurality of bodies 157, may fully cover each of the outer surfaces 187 of the plurality of bodies 157, and the like.

In some examples, the coating 132 includes metal such as silver, or thin silver plating which may be applied via deposition or other methods along the outer surfaces 187 to increase radiation attenuation and protect the healthy tissue. In some specific examples (FIG. 9C), each of the outer surfaces 187 is coated with the coating 132 where the coating 132 defines a silver sheet that provides 240 degrees of shielding. In many of these examples, the respective outer ends 188 of each of the plurality of bodies 157 may be devoid of the coating 132 to provide access to respective ones of the plurality of channels 114 for the radiation elements 156.

### Exemplary Materials

In some examples, the housing 112 of the device 102 may be formed with polyethylene (Low Density) (LDPE), the plurality of bodies 157 may be formed with polyurethane (PU), and the guidewire 116 may be formed with PU. The applicator cap 158 may be formed with LDPE, and the end cap 160 may be formed with thermoplastic polyurethane (TPU). The balloons 122 may be formed with polyethylene terephthalate (PET), and the balloon catheters 128 may be formed with PU. The housing 112 and the plurality of bodies 157 can included a length between 20-70 cm. The radiopaque markers 118 can be implemented using radiopaque ink. The end cap 160 and the applicator cap 158 can be applied to the distal portion 153B and the proximal portion 153A of the device 102 as described using a thermoplastic polyurethane injection modeled seal, and/or one or more of an adhesive. The foregoing is merely exemplary, and similar materials and combinations are contemplated.

### Exemplary Applications

During a brachytherapy procedure, the device 102 may be used in combination with other measures in order to treat cancer of the esophagus. An endoscopy, along with traditional imaging, may be used as one example to visualize a tumor and to allow for correct device placement. Dosimetry may further be completed by a medical physicist to determine optimal seed dwell time and locations. These calculations may then be programmed into the console for controlling the radioactive element (e.g., seed) during the treatment.

In other examples, the device 102 may be implemented to provide radiation treatment along other target sites such as a vaginal cavity, a rectum, and the like.

### Feasibility and Experimentation

### Radiation Analysis

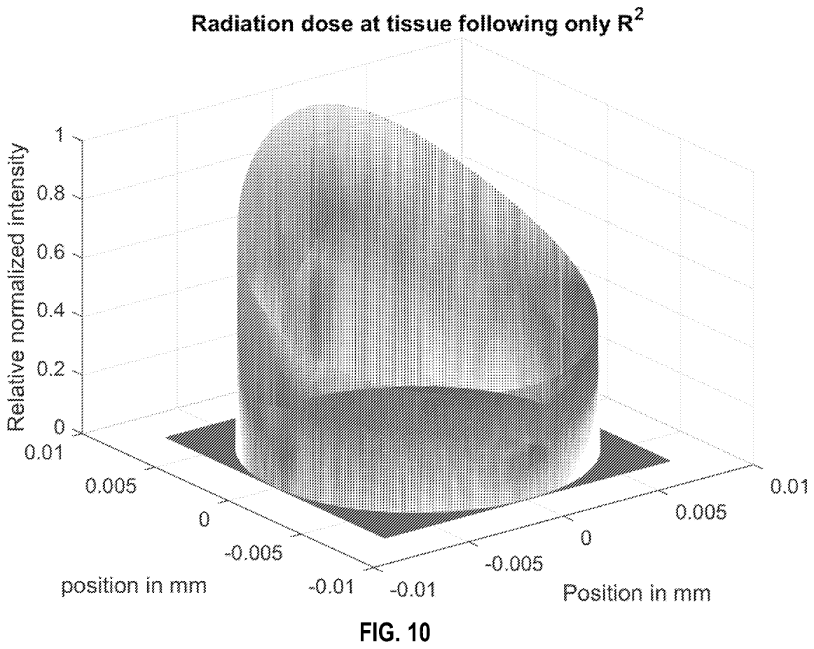

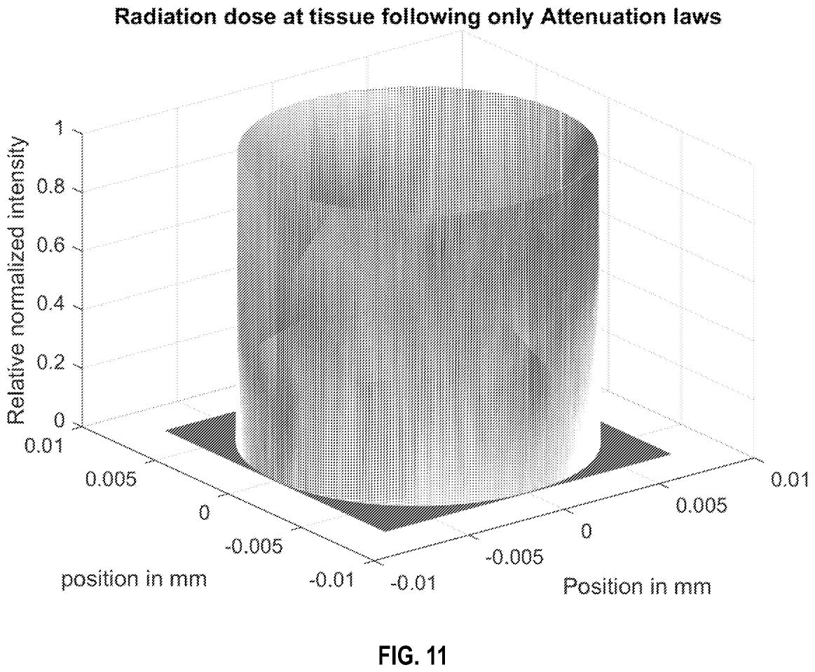

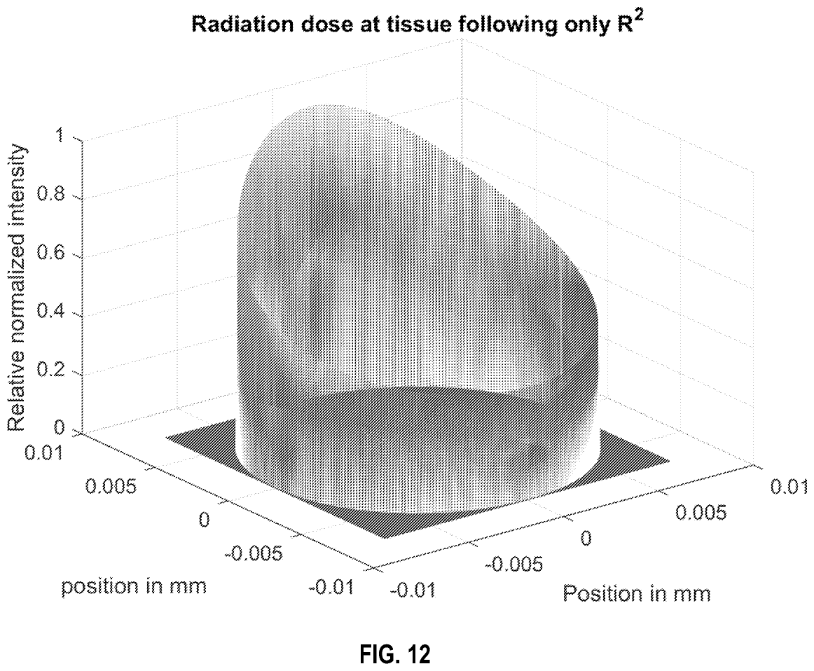

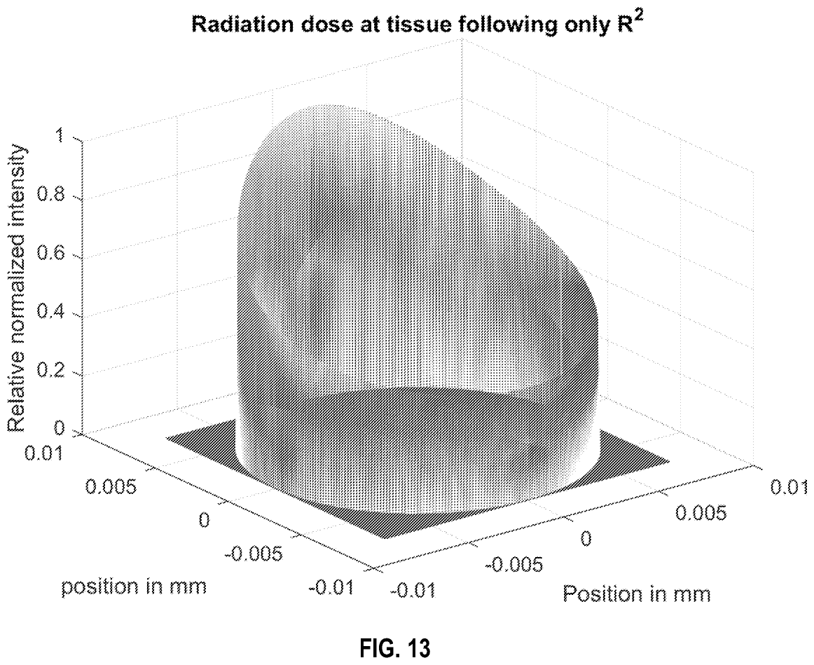

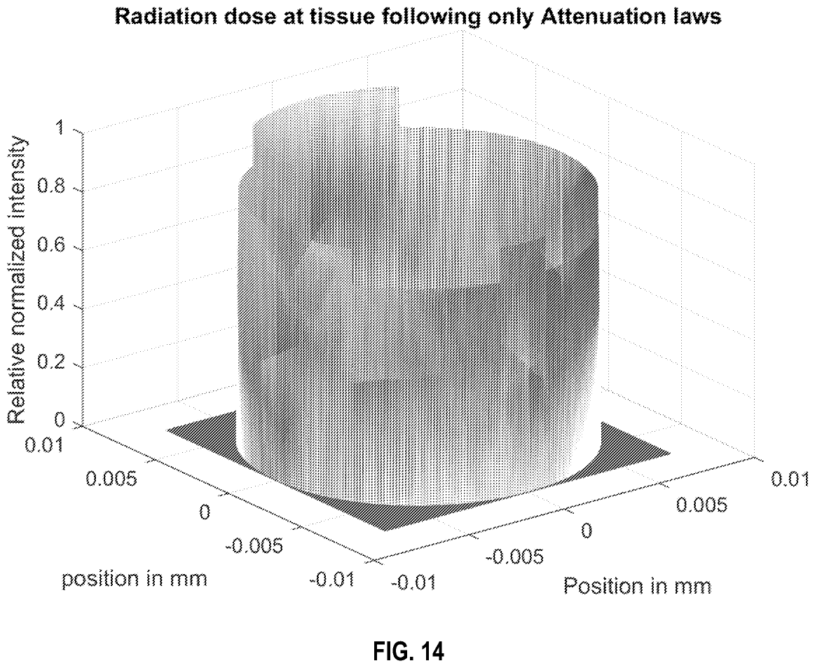

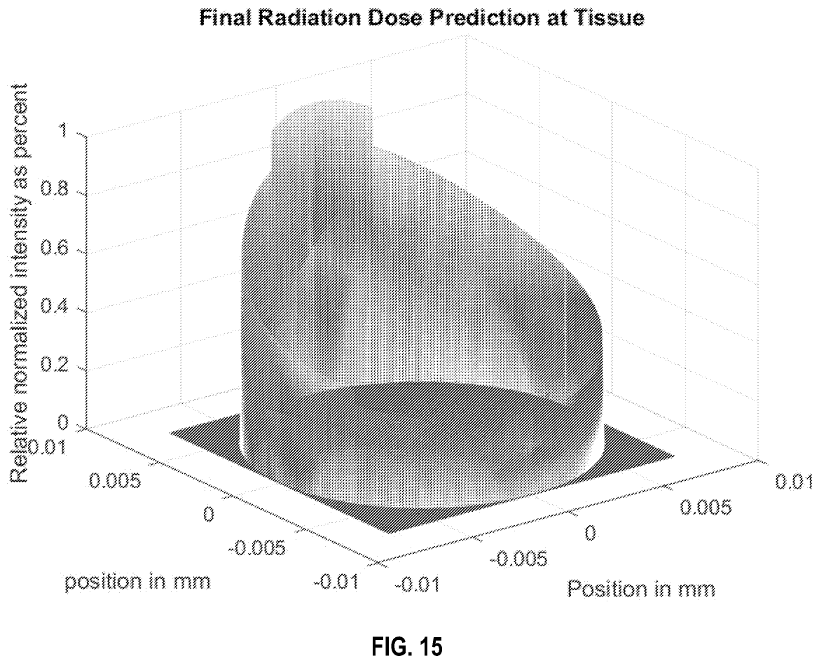

Various testing procedures were conducted to assess radiation dose applications using the device 102. In order to calculate relative radiation dose at the first layer of tissue, a MATLAB code was developed to automate the analysis of the device 102 and its feasibility. This allowed for parameters such as number of the plurality of channels 114, thickness of the plurality of channels 114, relative position of the plurality of channels 114, and thickness of attenuation materials to be inputted to project the dose profiles both graphically and numerically. With this code, it was determined that the preliminary design before a first engineering change request met a 2× dose to tumor relative to healthy tissue requirement, protecting 180 degrees of the modelled esophagus from excess radiation. After conducting more research into other attenuation methods and practices, the addition of a 12 μm silver plating coating (coating 132) to 240 degrees of all channels (114) allowed for the 2× radiation dose requirement to be optimized. With this enhanced analysis, 240 degrees of the modelled esophagus are protected from excess radiation. Graphs from this analysis can be found in FIGS. 10-15. These graphs show the radiation dose profiles around the esophagus due to the Inverse Square Law of Radiation alone, Beer's Law alone before and after the addition of Silver plating, and finally the combined finalized dose profile. Additionally, for comparative purposes, the finalized radiation dose profile of the predicate device can also be found.

### Sterilizability: Material Property Analysis

A material analysis was completed with assistance from the book Plastics in Medical Devices—Properties, Requirements, and Applications by Vinny R. Sastri. Forty-eight plastics were analyzed for their ability to undergo ethylene oxide sterilization, gamma radiation exposure, diluted acid exposure, biocompatibility for the esophagus, and for their ability to be made into a flexible plastic. This process allowed for the materials of each device part to be decided to follow the necessary requirements, finally resulting in a pass of the system requirement.

Two tests were completed to assess viability of the device 102, Applicator Flexibility and Safe Operation. The limit values are listed in Table 1: Test Verification Table, below.

TABLE 1
Test Verification Table

| | | | Measured/ | |
|---|---|---|---|---|
| | | Limit/ | Reference | Pass/ |
| Requirements | Method | Reference | Value | Fail |
| 4.3 Materials | | | | |
| 4.3.3 Applicator Flexibility: The | T | 10% +/− predicate | 2.43 cm | Pass |
| applicator shall match the flexibility of | | flexibility | | |
| the predicate device +/− ten percent. | | (2.34 cm, 2.86 cm) | | |
| 4.4 Safety | | | | |
| 4.4.1 Safe Operation: The device, | T | 0.5 cm +/− 0.5 cm | 0.0 cm | Pass |
| when balloons are inflated and held | | | | |
| vertically with a 75 g weight attached, | | | | |
| shall not move more than 0.5 cm +/− | | | | |
| 0.5 cm. | | | | |

### Applicator Flexibility Test

Introduction: This procedure outlines the acceptance test that was performed on the entire applicator subsystem 104. This test verified that the prototype device, which includes all subassemblies, has the flexibility properties comparable to the predicate device. The prototype device and the predicate device individually was fixed at both ends, leaving the center of each unsupported. A 75 g weight was hung from the center of both devices and the deflection of each device from their respective initial states was measured. It was determined that the prototype deflection distance at the center must equal that of the predicate device averages +/−10% for three trials in order to pass the verification test.

### Step-by-Step Procedure:

1. Distance the 2 platforms 36 cm apart from each other and fasten both the predicate device and the prototype device individually by their ends.
2. Locate the center of each device with a measuring tape and attach a 75 g weight to the center of each device using a rope/string.
3. Allow both of the devices to be pulled down at the center by the 75 g weight and note the deflection of both devices.
4. Measure the distance that the center of the predicate device has deflected from its initial position
5. Measure the distance that the center of the prototype has deflected from its initial position.
6. Repeat steps 1-5 two more times.
7. Average the deflection values of each device separately.
8. If the average distance travelled by the center of the prototype is equal to the distance travelled by the center of the predicate device, with a 10% difference allowable, then the prototype has passed the verification test.
9. If it has passed the verification test, the prototype device has comparable flexibility properties to the predicate device. Therefore by similarity, the flexibility of the prototype has been deemed to be within the sufficient range to be effective during the brachytherapy procedure. (Mark Pass/Fail on the datasheet.)

Results: A prototype of the device 102 deflected an average of 2.43 cm, a value within the predicate flexibility range. This resulted in a pass of the Applicator Flexibility test.

### Safe Operation Test

Introduction: The following procedure outlines an exemplary acceptance test that was performed upon the device 102. This test verifies that the prototype of the device 102, which includes all subassemblies, may be safely inserted and retracted in a patient and is intended (for some procedures) not move while in the patient. A prototype was inserted vertically into a model esophagus and then the stabilizing balloons were inflated. A known weight of 75 g was hung from the device's bottom to apply a constant force. It was determined that the prototype must move less than 1 cm for 3 trials in order to pass the subject verification test.

### Step-by-Step Procedure:

1) Insert prototype device into PVC pipe and inflate stabilizing balloons to desired level.
2) Mark start and end points of balloons relative to the PVC pipe.
3) Attach 75 g weight to the bottom of the prototype.
4) Hold the system vertical and hang weight for 30 seconds.
5) Mark the new start and end points of balloons relative to the PVC pipe.
6) Deflate balloons and remove the prototype.
7) Measure the difference between original markings and new markings.
8) If the distance is less than 1 cm, the device has passed the verification test. (Mark Pass/Fail on the datasheet.)
9) Repeat steps 1-8 two more times.

Results: The prototype device travelled an average of 0.0 cm along the modelled esophagus. This results in a pass of the Safe Operation test.

It should be understood that the foregoing testing procedures are merely exemplary and were not intended to and should not limit the scope of the present inventive concept. Rather, the testing procedures merely support the viability of the device 102 for real world applications.

### Exemplary Method

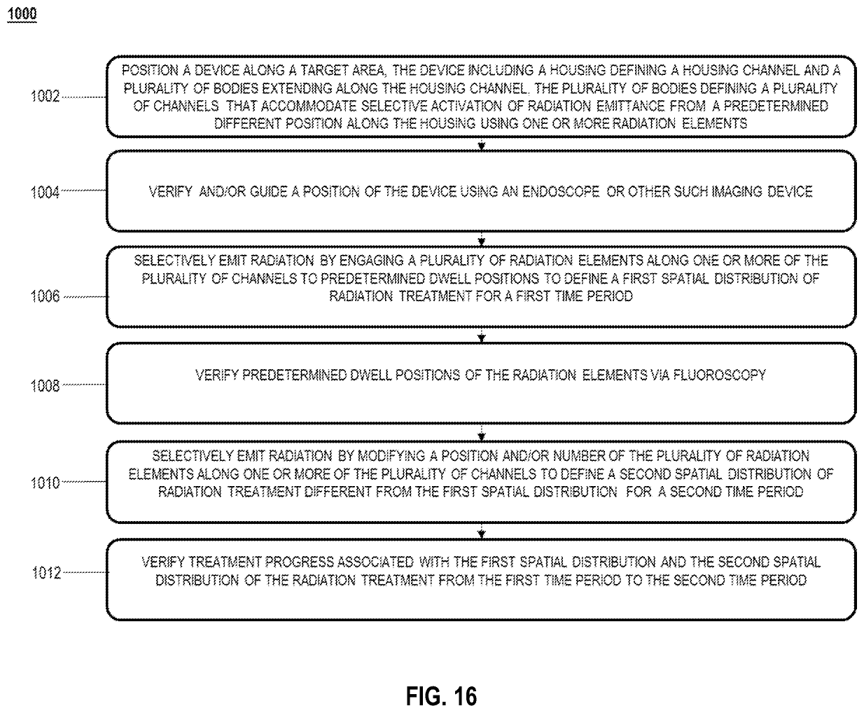

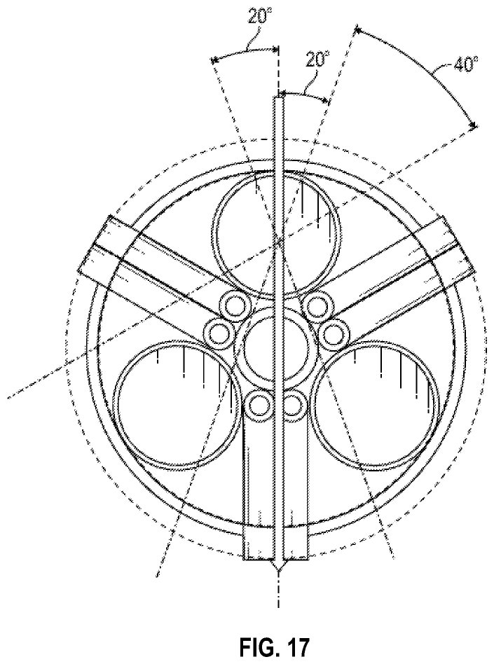

Referring to FIG. 16, a sample process or method 1000 associated with the system 100 including the device 102 shall now be described with reference to FIG. 17 and FIGS. 18A-18C and with continuing reference to FIGS. 3-4. As indicated in block 1002 and block 1004, the device 102 of FIGS. 3-4 can be positioned along a target area 200 (FIGS. 18A-18C), such as the esophagus, and a position of the device 102 can be verified using an endoscope 142 or other such imaging equipment. Examples of the device 102 include the housing 112 defining the housing channel 155, and the plurality of bodies 157 positioned within the housing 112 along the housing channel 155. The plurality of channels 114 defined by the plurality of bodies 157 accommodates selective activation of radiation emittance from a predetermined different position along the housing 112 using one or more of the radiation elements 156. In other words, the housing 112 is configured to receive at least one of the radiation elements 156 at a variable position along a predetermined channel of the plurality of channels 114.

As previously described and shown in FIG. 17, components of the device 102 within and along the housing 112 may be arranged in a symmetrical configuration. In some examples as shown, the plurality of bodies 157 are equidistantly arranged around the longitudinal axis 151 and the guidewire channel 117. In this manner, all of the plurality of channels 114 may produce the same radiation plot when fed with one or more radiation elements 156. In some examples, curvatures of the device 102 are simplified to two times the thickness. Multiplying the Inverse Square Law Intensity by the Beer's Law Intensity results in an overall intensity of the device 102.

Referring to block 1006 and block 1008 of FIG. 16 and to a first radiation phase 1800 of FIG. 18A (applying a first portion of radiation treatment to the target area 200), the device 102 may be implemented to selectively emit radiation by engaging one or more of the radiation elements 156 along one or more of the plurality of channels 114 of the plurality of bodies 157 to predetermined dwell positions along the housing 112 to define a first spatial distribution 1802 of radiation treatment that may be applied to the target area 200 for a first time period (predetermined amount of time). Specifically, for example, a radiation element 156A may be fed to a first channel 114A of the plurality of channels 114 and urged to a first predetermined position (1806A) (along the housing 112 and around the longitudinal axis 151), and a radiation element 156B may be fed to a second channel 114B of the plurality of channels 114 and urged to a second predetermined position (1806B) along the housing 112 and around the longitudinal axis 151. In other words, the first channel 114A is configured to receive the radiation element 156A along the first predetermined position 1806A and emit a first radiation plot 1808A along the predetermined position 1806A relative to the longitudinal axis 151, and the second channel 114B is configured to receive the radiation element 156B and emit a second radiation plot 1808B along the second predetermined position 1806B relative to the longitudinal axis 151. The first predetermined position 1806A and the second predetermined position 1806B may be aligned along a common horizontal plane relative to the longitudinal axis 151 (i.e., positioned a general common distance from the proximal portion 153A of the housing 112), or may be aligned along different positions relative to the proximal portion 153A of the housing 112. Notably, because the device 102 provides the first radiation plot 1808A and 1808B, the first spatial distribution 1802 can define shape configurations different from general concentric shapes for customizable dose delivery to the target area 200.

As indicated in block 1008, the position of the radiation element 156A and the radiation element 156B can be verified via a fluoroscopy device 154 or other such methods. The channel labels 120 can assist with the desired selection of the plurality of channels 114 and engagement of the radiation elements 156. In some examples, the radiopaque markers 118 can further assist with positioning of the radiation elements 156.

Referring to block 1010, and referencing a second radiation phase 1820 of FIG. 18B (applying a second portion of radiation treatment to the target area 200), the device 102 may be implemented to selectively emit radiation by engaging one or more of the radiation elements 156 along one or more of the plurality of channels 114 of the plurality of bodies 157 to predetermined dwell positions along the housing 112 to define a second spatial distribution 1822 of radiation treatment that may be applied to the target area 200 for a second time period (predetermined amount of time). Specifically, for example, the radiation element 156A may be fed to the first channel 114A of the plurality of channels 114 and urged to the first predetermined position (1806A) along the housing 112 and around the longitudinal axis 151, the radiation element 156B may be fed to the second channel 114B of the plurality of channels 114 and urged to the second predetermined position (1806B) along the housing 112 and around the longitudinal axis 151, and in addition, a radiation element 156C may be fed to a third channel 114C of the plurality of channels 114 and urged to a third predetermined position (1806C) along the housing 112 and around the longitudinal axis 151. In other words, the first channel 114A is configured to receive the radiation element 156A along the first predetermined position 1806A and emit the first radiation plot 1808A along the predetermined position 1806A relative to the longitudinal axis 151, the second channel 114B is configured to receive the radiation element 156B and emit the second radiation plot 1808B along the second predetermined position 1806B relative to the longitudinal axis 151, and the third channel 114C is configured to receive the radiation element 156C and emit the third radiation plot 1808C along the third predetermined position 1806C relative to the longitudinal axis 151. In this manner, the first radiation plot 1808A, the second radiation plot 1808B, and the third radiation plot 1800C collectively define the second spatial distribution 1822 which is greater in size than the first spatial distribution 1802 by nature of the implementation of an additional radiation element (156C).

As indicated by the third exemplary radiation phase 1840 in FIG. 18C, radiation emittance from the device 102 can be selectively reduced as desired. Specifically, for example, the radiation element 156B may be fed to the second channel 114B of the plurality of channels 114 and urged to the second predetermined position (1806B) along the housing 112 and around the longitudinal axis 151 to emit the second radiation plot 1808B, while the first channel 114A and the third channel 114C may be devoid of radiation elements (or the radiation element 156A and the radiation element 156C may be removed).

As noted in block 1012 of FIG. 16, treatment progress associated with the first phase of radiation treatment, the second phase of radiation treatment, and the third phase of radiation treatment can be verified, tracked, or monitored or otherwise using any form or combination of imaging equipment, including dosimetry. For example, where the target area 200 includes a cancerous tumor, such equipment and methods can be implemented to monitor efficacy of the radiation treatment applied by the device 102 to the target area from the first time period to the second time period, and so on.

It should be understood from the foregoing that, while particular examples have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system for precision radiation treatment, comprising:
a device associated with an endoscopy guided procedure, comprising:
a housing defining a housing channel extending along a longitudinal axis of the housing, at least a portion of the housing configured for deployment within a cavity;
a plurality of bodies defining a plurality of channels extending through the housing and defined around the longitudinal axis, wherein each of the plurality of channels accommodates selective activation of radiation emittance from a predetermined different position along the housing via one or more radiation elements such that the plurality of channels collectively provides a predetermined spatial distribution of a radiation treatment to a target area along the cavity; and
a stabilizing subsystem that stabilizes the housing along the cavity.

2. The system of claim 1, further comprising an endoscope, wherein the endoscope assists with endoscopy-guided placement of the device along a gastrointestinal tract.

3. The system of claim 1, wherein the predetermined spatial distribution of the radiation treatment defines a plurality of shape configurations different from general concentric shapes for customizable dose delivery.

4. The system of claim 1, wherein the plurality of channels comprises a first channel configured to receive a first radiation element and emit radiation along a first position relative to the longitudinal axis of the housing and a second channel of the plurality of channels configured to receive a second radiation element and emit radiation along a second position relative to the longitudinal axis.

5. The system of claim 1, further comprising a fluoroscopy subsystem that verifies a predetermined position of one or more radiation elements along one or more of the plurality of channels.

6. The system of claim 1, wherein the housing and the plurality of channels collectively define an applicator subsystem, the applicator subsystem configured to:
apply a first portion of the radiation treatment according to a first spatial distribution configuration along the target area during a first time period, and
apply a second portion of the radiation treatment according to a second spatial distribution configuration along the target area during a second time period.

7. The system of claim 1, wherein the housing is configured to receive a radiation element and urge the radiation element through a selected channel of the plurality of channels.

8. The system of claim 1, wherein the housing is configured to receive at least one radiation seed at a variable position along a predetermined channel of the plurality of channels.

9. The system of claim 1, wherein the housing comprises a flexible material such that the housing accommodates a predetermined amount of deflection between a proximal portion of the housing and a distal portion of the housing.

10. The system of claim 1, further comprising an applicator cap that engages to a proximal portion of the housing, the applicator cap including a plurality of applicator channels configured for alignment with the plurality of channels of the housing to feed one or more radiation elements through the plurality of applicator channels to the plurality of channels for the radiation treatment.

11. The system of claim 1, wherein the stabilizing subsystem includes:
one or more balloon catheters formed within the housing along the longitudinal axis and one or more balloons positioned along the housing, each balloon catheter of the one or more balloon catheters being configured for fluid flow communication with a respective balloon, the one or more balloons being positioned exterior to a distal portion of the housing to stabilize the housing along the cavity.

12. The system of claim 11, wherein each balloon catheter of the one or more balloon catheters includes a respective valve for intake and expulsion of air from a balloon.

13. The system of claim 1, further comprising a metal coating applied along outer surfaces of the plurality of channels of the housing that attenuates radiation and protects healthy tissue proximate to the target area.

14. The system of claim 1, further comprising a plurality of radiopaque markers defined along the housing that guide a physician to position the housing under a CT scan.

15. The system of claim 1, further comprising a guidewire extending along the longitudinal axis of the housing that provides assistance with guiding the housing to a predetermined position relative to the cavity.
